(19) United States
(12) Patent Application Publication
(10) Pub. No.: US 2026/0227244 A1
(43) Pub. Date: Aug. 6, 2026

(54) WIRELESS FOOD TEMPERATURE PROBE WITH MOISTURE DETECTION FUNCTION

(71) Applicant: Shenzhen TOPOS Sensor Technology Co., LTD., Shenzhen (CN)

(72) Inventors: ZHAOTING ZENG, Shenzhen (CN); HUI ZHANG, Shenzhen (CN)

(21) Appl. No.: 19/635,017

(22) Filed: Mar. 31, 2026

(30) Foreign Application Priority Data
Mar. 17, 2026 (CN) 202620328172.X

(51) Int. Cl. G01K 1/024; G01K 1/02; G01K 7/02; G01N 27/22; G01N 33/02

(52) U.S. Cl. CPC G01K 1/024; G01K 1/026; G01K 7/02; G01N 27/223; G01N 33/02; G01K 2215/00

(57) ABSTRACT

Provided is a wireless food temperature probe with moisture detection function, which includes a probe body and a circuit board component. The probe body includes a metal tip component, a metal tube component, and a first insulation connector. The metal tip component and the metal tube component are connected through the first insulation connector. The metal tip component is provided with a first temperature sensor for detecting an internal temperature of food and is electrically connected to the circuit board component. The metal tip component and the metal tube component are used as two electrode plates for detecting capacitors and are connected to the circuit board component. The moisture content of food is obtained by detecting a capacitance value between the metal tip component and the metal tube component. The probe shell is used as a detection electrode, without the need for additional moisture sensors.

### CROSS-REFERENCE TO RELATED APPLICATIONS

This application claims priority to Chinese Patent Application No. 202620328172.X, filed on March 17, 2026, which is hereby incorporated by reference in its entirety.

### TECHNICAL FIELD

The present disclosure relates to the field of intelligent food temperature probe technologies, and in particular, to a wireless food temperature probe with moisture detection function.

### BACKGROUND

With the popularity of smart kitchenware, wireless food temperature probes are widely used in cooking equipment such as ovens and brasiers to monitor the internal temperature of meat and other foods in real time. In order to avoid damaging the organizational structure of food, preserve food juice, and facilitate operation, the physical size of food probes is greatly limited, with a diameter typically only a few millimeters.

High quality cooking not only requires temperature data, including the temperature inside the food and the ambient temperature, but also in many scenarios, it is necessary to detect moisture content inside the food (including the degree of moisture loss). Due to the extremely small internal space of the food temperature probe, existing probes cannot add independent moisture sensors, resulting in a single function that cannot meet advanced cooking needs.

Therefore, there is an urgent need for a new probe structure in the field of food probes to achieve efficient and safe multi parameter detection of internal temperature and moisture in food without increasing the probe volume.

### SUMMARY

Based on this, it is necessary to propose a wireless food temperature probe with moisture detection function.

A wireless food temperature probe with moisture detection function, including: a probe body and a circuit board component that is provided in the probe body;

the probe body includes a metal tip component, a metal tube component, and a first insulation connector; the metal tip component and the metal tube component are connected and electrically isolated through the first insulation connector;

the metal tip component is provided with a first temperature sensor configured to detect an internal temperature of food, and the first temperature sensor is electrically connected to the circuit board component;

the metal tip component and the metal tube component serve as two electrode plates for detecting capacitors, respectively; the circuit board component is electrically connected to the metal tip component and the metal tube component, respectively; the circuit board component obtains moisture content of food by detecting a capacitance value between the metal tip component and the metal tube component.

In some embodiments of the present disclosure, the circuit board component includes a first temperature measurement module and a moisture detection module, the first temperature sensor is electrically connected to the first temperature measurement module, and the moisture detection module is electrically connected to the metal tip component and the metal tube component respectively to obtain the moisture content of the food.

In some embodiments of the present disclosure, the moisture detection module is further configured to obtain a salinity of the food based on the capacitance value.

In some embodiments of the present disclosure, the probe body further includes a second insulation connector and a metal tail component; the metal tube component and the metal tail component are connected and electrically isolated through the second insulation connector.

In some embodiments of the present disclosure, the metal tip component, the first insulation connector, the metal tube component, the second insulation connector, and the metal tail component are sequentially connected from front to back to form the probe body.

In some embodiments of the present disclosure, the metal tail component is provided with a second temperature sensor configured to detect an ambient temperature, and the circuit board component further includes a second temperature measurement module, where the second temperature sensor is electrically connected to the second temperature measurement module.

In some embodiments of the present disclosure, the metal tail component is configured as a second temperature sensor carrier, the second temperature sensor is a thermocouple, a hot end of the thermocouple is connected to the metal tail component to render the metal tail component as a part of a thermocouple circuit, and a cold end of the thermocouple is connected to the second temperature measurement module of the circuit board component through a first wire and a second wire.

In some embodiments of the present disclosure, the metal tail component is configured as an antenna for wireless signal transmission, and the circuit board component further includes an Radio Frequency, (RF) main control chip and a time-sharing circuit, the time-sharing circuit is respectively electrically connected to the RF main control chip and the second temperature measurement module to achieve time-sharing processing of wireless signal transmission and environmental temperature acquisition.

In some embodiments of the present disclosure, the metal tail component is configured as a part of an external charging circuit, and when charging externally, the metal tip component or the metal tube component serves as a first charging electrode of the external charging circuit, and the metal tail component serves as a second charging electrode of the external charging circuit, to charge a battery built into the probe body through the circuit board component.

In some embodiments of the present disclosure, the circuit board component further includes an interface multiplexing circuit; the interface multiplexing circuit at least includes an isolation capacitor and a RF choke inductor; the RF main control chip is electrically connected to the metal tail component through the isolation capacitor, and the second temperature measurement module is electrically connected to the metal tail component through the RF choke inductor to achieve physical isolation between a RF signal and an environmental temperature acquisition signal.

In some embodiments of the present disclosure, the circuit board component further includes a charging module and a switching circuit, the charging module is electrically connected to the battery; the metal tail component is connected to the charging module through the switching circuit, and the switching circuit is configured to disconnect an electrical connection between the metal tail component with an internal temperature measurement path and a signal transmission path when the metal tail component is connected to an external power source.

Compared with the existing technology, the beneficial effect of the present disclosure is that it divides the metal shell of a first half of the probe body into the metal tip component and the metal tube component, and uses the first insulation connector for electrical isolation, so that these two natural metal shells are directly converted into the two electrode plates of the detection capacitor. There is no need to add any additional physical moisture sensor components inside the probe. By detecting the capacitance value between the two, the moisture content and even salinity of the food can be accurately obtained, greatly saving internal space and reducing assembly complexity.

### BRIEF DESCRIPTION OF DRAWINGS

In order to provide a clearer explanation of the embodiments or technical solutions in the present application or existing technology, a brief introduction will be given to the accompanying drawings required for the embodiments or existing technology description. It is obvious that the accompanying drawings described below are only some embodiments of the present application. For those skilled in the art, other drawings can be obtained based on these drawings without creative work.

FIG. 1 is a schematic structural diagram of a food temperature probe in an embodiment.

FIG. 2 is a schematic diagram of various functions of a metal shell of the food temperature probe in an embodiment.

FIG. 3 is a schematic diagram of a connection between each module of a circuit board component of the food temperature probe and the metal shell in an embodiment.

FIG. 4 is a schematic diagram of a circuit connection of the food temperature probe compatible with food internal temperature detection, food internal moisture detection, and environmental temperature detection functions in an embodiment.

FIG. 5 is a schematic structural diagram of a thermocouple of the food temperature probe in an embodiment.

FIG. 6 is a schematic diagram of a circuit connection of the food temperature probe compatible with internal temperature detection, internal moisture detection, and wireless signal transmission of an antenna in an embodiment.

FIG. 7 is a schematic diagram of a circuit connection of the food temperature probe configured with a charging function in an embodiment.

Numeral reference: 11- metal tip component; 12- metal tube component; 13- first insulation connector; 14- metal tail component; 15- second insulation connector; 21- first temperature sensor; 22- battery; 23- circuit board component; 24- second temperature sensor.

### DESCRIPTION OF EMBODIMENTS

In order to enable those skilled in the art to better understand the technical solutions in this application, the following will provide a clear and complete description of the technical solutions in the embodiments of this application in combination with the accompanying drawings. Obviously, the described embodiments are only a part of the embodiments of this application, not all of them. Based on the embodiments in this application, all other embodiments obtained by those skilled in the art without creative work are within the protection scope of this application.

### Embodiment 1

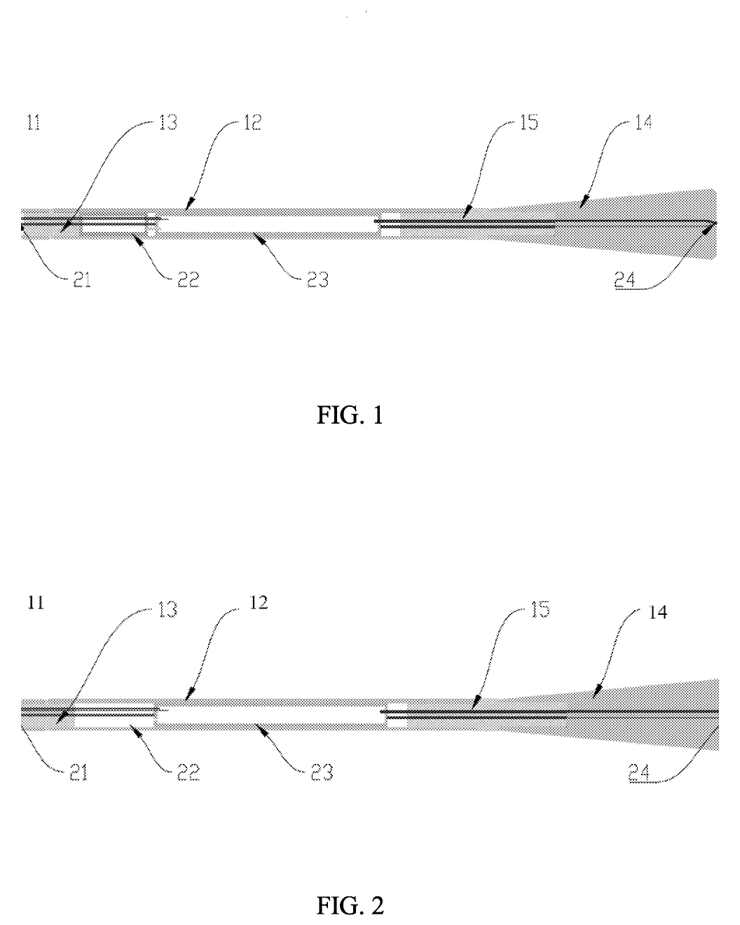

As shown in FIG. 1, this embodiment provides a wireless food temperature probe with moisture detection function. This wireless food temperature probe is mainly used in cooking equipment such as ovens and brasiers, and can monitor a plurality of parameters such as internal temperature of food (such as grilled meat), external environment furnace temperature, and moisture content inside the food in real time without damaging the food tissue structure. The wireless food temperature probe generally includes a probe body and a core control unit, namely a circuit board component 23, which is enclosed and built into the probe body, as well as a first temperature sensor, a second temperature sensor, a battery, and connection wires.

In order to achieve a plurality of detection functions in extremely narrow pipe diameter space, the probe body in this embodiment adopts a segmented isolation design in physical form. Specifically, as shown in FIG. 1, the shell of the probe body includes, from front to back, a metal tip component 11, a first insulation connector 13, a metal tube component 12, a second insulation connector 15, and a metal tail component 14. In an implementation mode, as shown in FIG. 2, the metal tip component 11 is specifically a stainless steel tip, the metal tube component 12 is specifically a stainless steel tube, and the metal tail component 14 is specifically a tail stainless steel part.

In terms of assembly relationship, the metal tip component 11 and the metal tube component 12 are fixedly connected through the first insulation connector 13, and a strict electrical isolation is achieved in terms of electrical performance. Similarly, the metal tube component 12 and the metal tail component 14 are fixedly connected through the second insulation connector 15 and achieve an electrical isolation.

In an implementation mode, the first insulation connector 13 and the second insulation connector 15 are made of high-temperature resistant special engineering plastics or ceramic materials, which not only ensure electrical disconnection between each metal segment, but also guarantee the overall mechanical compressive strength and internal waterproof and vapor sealing performance of the probe in high-temperature and high humidity cooking environments.

The probe body is internally formed with an accommodation cavity for precise arrangement of various electronic components. As shown in FIG. 1, an inner bottom of the metal tip component 11 is provided with a first temperature sensor 21 for detecting an internal temperature of food, in order to quickly sense temperature changes in a center of the meat. The circuit board component 23 and the battery 22 that supplies power to the circuit board component 23 are built into the accommodation cavity of the probe body. In addition, an interior or a surface of the metal tail component 14 is further provided with a second temperature sensor 24 configured to detect an ambient temperature of the oven. The first temperature sensor 21, the battery 22, and the second temperature sensor 24 are all electrically connected to the circuit board component 23.

As shown in FIG. 2, FIG. 2 is a schematic diagram of various functions of a metal shell of the food temperature probe in an embodiment. The core innovative concept of the present disclosure lies in an electronic componentization of the shell structural components, which breaks the technical bias of traditional probe metal shells only serving as physical protective layers. In front structure, the metal tip component 11 and the metal tube component 12, which have been physically divided and insulated, are directly endowed with the electrical function of detecting capacitor plates. In a later structure, the metal tail component 14 is endowed with a plurality of composite electrical functions, including a wireless signal transmission antenna, a carrier for environmental temperature measurement thermocouples (i.e. a part of a thermocouple circuit), and a charging electrode of an external charging circuit. This highly integrated interface and structural reuse design enables this probe to be compatible with multi parameter and multi-channel comprehensive detection and communication while maintaining the size of the ultra-thin tube.

### Front end reuse and moisture detection principle

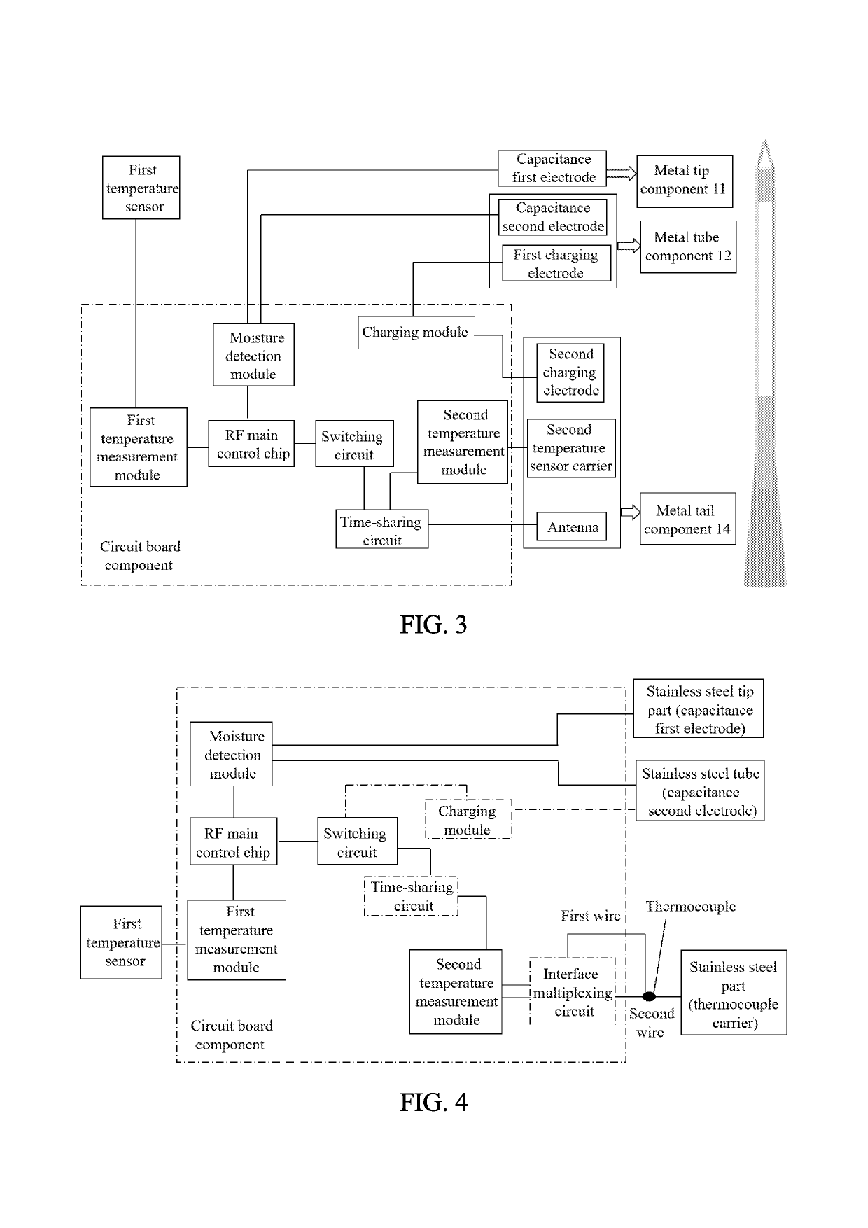

As shown in FIG. 3, FIG. 3 is a schematic diagram of a connection between each module of a circuit board component of the food temperature probe and the metal shell in an embodiment. In this embodiment, in order to achieve precise monitoring of the food state, the circuit board component 23 is highly integrated with a first temperature measurement module and a moisture detection module. On the basic electrical connection topology, an input terminal of the circuit board component 23 has established multi-channel signal connections with the metal shell at a front end of the probe body and internal sensors.

In an implementation mode, please refer to FIG. 4, which is an internal circuit connection diagram of this temperature probe when it is compatible with three functions: food internal temperature detection, food internal moisture detection, and environmental temperature detection. As shown on a left side of FIG. 4, when collecting meat temperature inside food, the first temperature sensor 21 built into the metal tip component 11 is electrically connected to the first temperature measurement module in the circuit board component 23. When the probe body pierces into the interior of grilled meat or other food, the first temperature sensor 21 senses real-time changes in the center temperature of the food and transmits weak resistance or voltage change signals to the first temperature measurement module for analysis, thereby obtaining internal temperature data of the food.

In an implementation mode, in order to obtain the moisture loss of food without increasing the probe diameter and internal physical sensing devices, the present disclosure directly reuses the metal shell in the front section of the probe as a moisture detection component. As shown in FIG. 4, due to the physical disconnection and electrical isolation achieved by the first insulation connector 13 between the metal tip component 11 and the metal tube component 12, these two suspended conductive metal bodies naturally form two electrode plates of a detection capacitor in physics.

Based on this structure, the moisture detection module inside the circuit board component 23 is electrically connected to the metal tip component 11 and the metal tube component 12, respectively. When the probe penetrates the food, the mixed medium of meat juice, oil, and moisture inside the food acts as a dielectric between the electrode plates. The moisture detection module detects an equivalent capacitance value between the two metal shells in real time by applying specific detection signals to the metal tip component 11 and the metal tube component 12. Due to the significant change in dielectric constant caused by moisture loss during different baking stages of food, the circuit board component 23 can calculate and obtain the current moisture content of the food based on the change in capacitance value.

In an implementation mode, after obtaining the capacitance values mentioned above, the moisture detection module can also be configured to further obtain the salinity of the food based on a specific dielectric constant algorithm model, thereby providing richer fire judgment data for high-order cooking.

It should be emphasized that the above-mentioned processes of measuring meat temperature and moisture content by the electrode plates occur in the first half structure of this insulation section. Due to the non-interference of their detection mechanisms and the physical isolation of the second insulation connection 15 from the metal tail component 14 in the second half, the multi parameter acquisition in the first half can be carried out simultaneously with other working states such as signal transmission in the second half of the probe, thereby greatly improving the data acquisition efficiency of the system.

Tail structure and environmental temperature acquisition principle

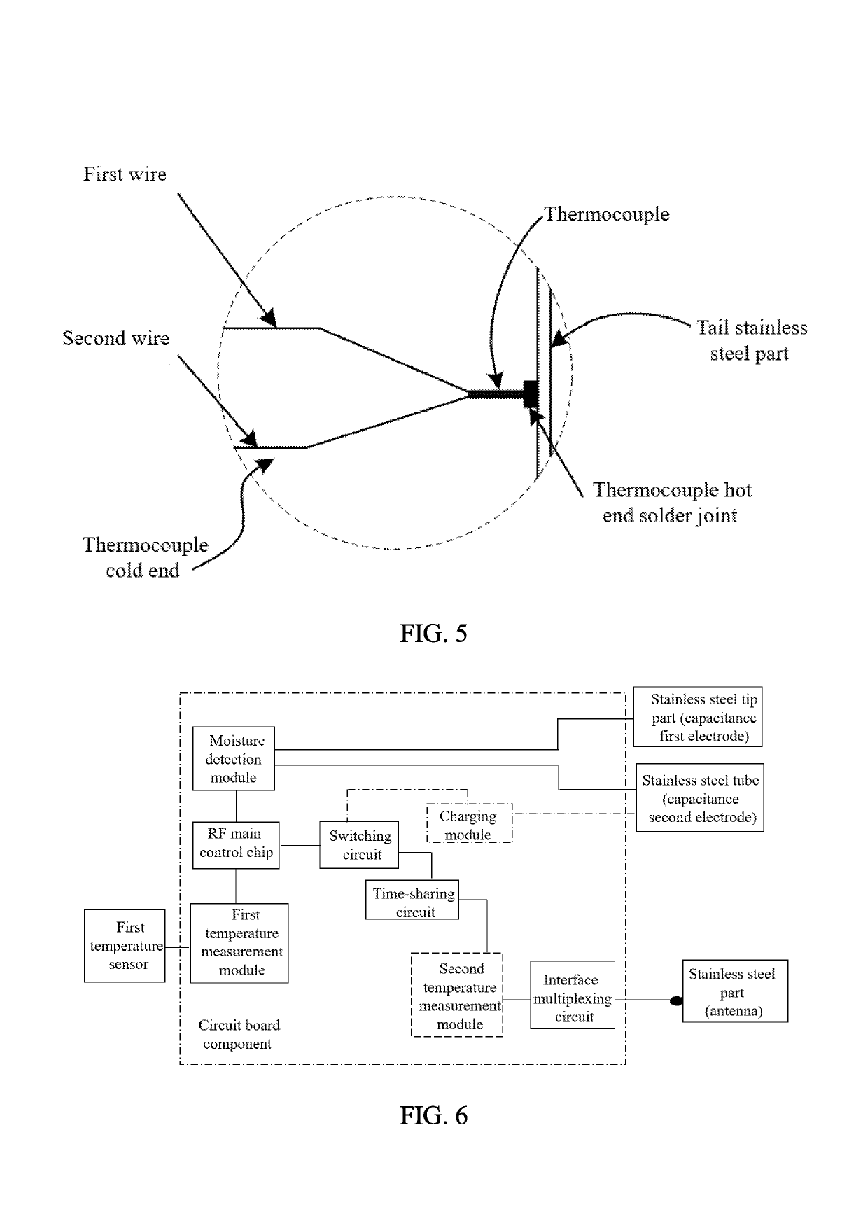

As shown in FIG. 5, FIG. 5 is a schematic structural diagram of a thermocouple for the food temperature probe in an embodiment. In order to monitor an internal temperature of the oven in real time, the present disclosure is provided with a second temperature sensor 24 at one end of the probe. In an implementation mode, the second temperature sensor 24 is specifically a thermocouple.

The thermocouple consists of a first wire and a second wire, the two wires are connected at one end to form a hot end, i.e. a measuring end, and at the other end to form a cold end, i.e. a reference end.

Combined with FIG. 4, this embodiment has redesigned the physical wiring and fixing method of the thermocouple. In this structure, the metal tail component 14 is configured as a temperature sensing carrier for the second temperature sensor 24. Specifically, the hot end of the thermocouple is directly physically connected to an inner wall or a surface of the metal tail component 14. This structural design makes the metal tail component 14 no longer just a mechanical protective shell, it directly becomes a part of a thermocouple measurement circuit in electrical topology.

On this physical basis, the cold end of the thermocouple extends inward through the first wire and the second wire, crosses the second insulation connector 15, and finally connects to the second temperature measurement module of the circuit board component 23.

In a pure ambient temperature detection working state shown in FIG. 4, when the high-temperature environment inside the oven heats the metal tail component 14 exposed to the outside of the food, due to the material difference between the two wires of the thermocouple, an extremely weak Direct Current, (DC) temperature difference electromotive force will be generated inside. This weak DC temperature measurement signal passes through the internal wiring path along the first and second wires, and finally flows into the second temperature measurement module inside the circuit board component 23. The second temperature measurement module performs high-precision amplification and analog-to-digital conversion on the weak DC voltage signal to calculate the current oven ambient temperature.

This design, which directly parasitizes the hot end of the thermocouple on the metal tail shell, not only makes the probe respond extremely quickly to external environmental temperature, but also maximizes the saving of the narrow internal space at the tail.

Tail end RF antenna reuse and multiplexing isolation mechanism

On the premise of having its technical advantages, the solution of Embodiment 3 further poses a significant electromagnetic interference hazard when reusing the metal tail component 14 as an RF antenna.

As shown in FIG. 6, FIG. 6 is a schematic diagram of a circuit connection of the food temperature probe compatible with internal temperature detection, internal moisture detection, and wireless signal transmission of an antenna in an embodiment.

In order to achieve data exchange between the probe and external intelligent terminals, this embodiment further configures the metal tail component 14 as an antenna for wireless signal transmission. When the probe body penetrates the interior of the food, the metal tail component 14 is exposed in a furnace cavity outside the food, thereby becoming a radio frequency electromagnetic wave radiator.

However, at this time, the metal tail component 14 and internal wires will simultaneously carry two types of electrical signals: one is the high-frequency, high-energy RF antenna signal emitted by the RF control chip, and the other is the extremely weak DC temperature difference signal generated by the thermocouple. If these two signals are transmitted simultaneously on the same physical path, high-frequency RF energy will instantly overwhelm the weak temperature measurement signal, and may even directly penetrate the fragile second temperature measurement module.

In order to solve this technical problem, the present disclosure introduces a dual isolation mechanism of hardware impedance capacitance shunt and software time-division scheduling in the circuit board component 23.

Firstly, in terms of hardware physical isolation, please compare FIGS. 4 and 6. The circuit board component 23 not only includes an RF main control chip, but also has an interface multiplexing circuit specially designed. The interface multiplexing circuit utilizes the filtering characteristics of basic electronic components, which at least include an isolation capacitor and a RF choke inductor. In this cross network, the RF main control chip is electrically connected to the metal tail component 14 (as an antenna) through the isolation capacitor. At the same time, the second temperature measurement module is electrically connected to the metal tail component 14 (as a thermocouple carrier) through the RF choke inductor.

During operation, the isolation capacitor utilizes its physical characteristics of high-frequency conduction and DC resistance to open the transmission channel for high-frequency signals emitted by the RF main control chip, allowing them to directly reach the metal tail component 14 for spatial radiation. On the contrary, the RF choke inductor exhibits its physical characteristics of conducting DC and blocking high-frequency, allowing the weak DC voltage signal of the thermocouple to pass smoothly, and blocking the high-frequency RF signal from the entrance of the second temperature measurement module, thereby achieving reliable shunting of high and low frequency signals at the physical interface.

Secondly, at the software isolation level of logic control, although there are hardware barriers, to pursue maximum temperature measurement accuracy and signal purity, the circuit board component 23 in this embodiment is further provided with a time-sharing circuit. The time-sharing circuit is electrically connected to the RF main control chip and the second temperature measurement module, thereby serving as a control node for the entire interface multiplexing system.

In an implementation mode, on the microscopic timeline, the time-sharing circuit forces the system to switch rapidly between the ambient temperature acquisition state shown in FIG. 4 and the wireless signal transmission state shown in FIG. 6, to achieve time-sharing processing.

During the temperature measurement period (as shown in FIG. 4), the time-sharing circuit controls the RF main control chip to sleep or remain silent, and reads the pure DC signal passing through the choke inductor through the second temperature measurement module. During a transmission period (state shown in FIG. 6), the time-sharing circuit suspends the sampling action of the second temperature measurement module, calls the RF main control chip, and converts packaged internal temperature, moisture content, and environmental furnace temperature data of the system into high-frequency RF signals, which pass through the isolation capacitor and broadcast to the external network through the metal tail component 14.

This soft hard combination scheme of bottom layer impedance capacitance shunt and upper layer time-division and multiplexing overcome the electromagnetic crosstalk problem of high and low frequency signal transmission, allowing the extremely limited probe tail space to unleash enormous multidimensional application potential.

Tail end charging reuse and switching protection mechanism

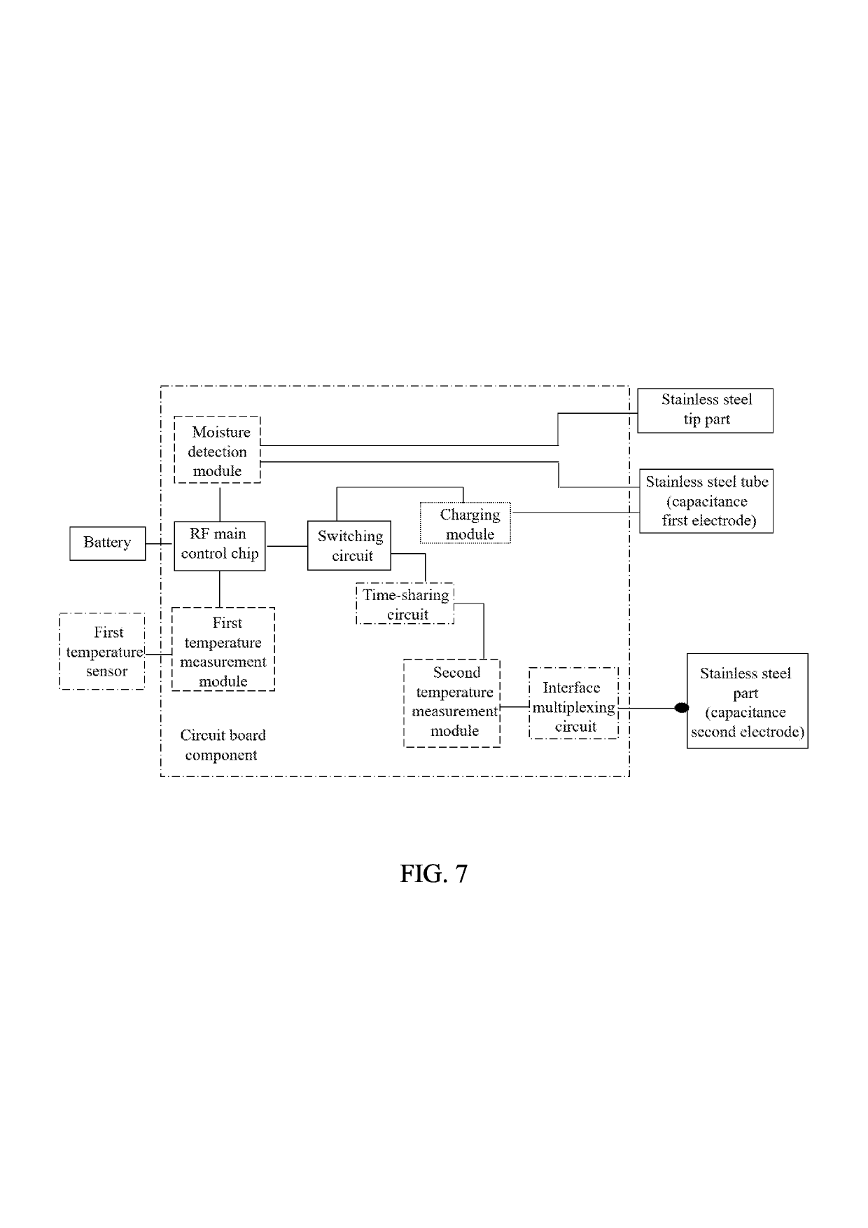

As shown in FIG. 7, FIG. 7 is a schematic diagram of a circuit connection of the food temperature probe configured with a charging function in an embodiment.

In order to increase long-term wireless endurance of the temperature probe and further extract the reuse value of the external metal shell, the present disclosure continues to integrate the charging function of the charging module based on the existing hardware structure.

As shown in FIG. 7, the metal tail component 14 is configured as a part of an external charging circuit. When the probe body is used up, the probe body is pulled out from food as a whole and placed on a matching external charging base (not shown in the figure), the metal shell outside the probe directly serves as a charging contact. Specifically, the metal tail component 14 serves as a second charging electrode (e.g. positive electrode) of the external charging circuit, and the metal tip component 11 or the metal tube component 12 in the front section serves as a first charging electrode (e.g. negative electrode or system ground) of the external charging circuit. The external charging current is introduced into the interior of the probe through the metal components, to charge the battery 22 built into the probe body through the circuit board component 23. It is worth noting that the moisture detection module, charging module, first temperature measurement module, and second temperature measurement module are circuit structures arranged on the RF main control chip.

However, this external structural reuse poses a serious security threat to internal circuits. As mentioned earlier, the metal tail component 14 is simultaneously connected to the fragile thermocouple, the high-precision RF control chip, and the second temperature measurement module. When the probe is connected to the external charging base, the external power will instantly flow in along the metal tail component 14 and its internal wires. If this high-energy charging current is not blocked, the internal precision temperature measurement and communication links will be instantly broken down by overvoltage or even burned out.

In order to solve this technical problem, combined with the circuit topology shown in FIG. 7, the circuit board component 23 is further provided with a charging module and a switching circuit at an access end. An output terminal of the charging module is electrically connected to the battery 22 to manage the charging process; and the switching circuit serves as the circuit switching core of the entire system.

The metal tail component 14 is connected to the charging module through the switching circuit. The switching circuit is configured with a strict conditional triggering mechanism: when it detects that the metal tail component 14 is connected to an external power source (i.e., the probe is inserted into a charging dock, as shown in the state of FIG. 7), the switching circuit instantly acts, forcibly disconnecting the electrical connection between the metal tail component 14 and the internal temperature measurement path and signal transmission path. At this point, the external high current is precisely directed to the safe charging module by the switching circuit.

Through this hard cut-off mechanism, the present disclosure establishes a safety logic of charging and operation being mutually exclusive in the physical circuit.

Sleep charging state (FIG. 7): the probe is in the base, and the switching circuit is activated. The sensitive working circuit inside is completely physically isolated, and the system only performs safe closed-loop charging.

Parallel working state (FIG. 4 and FIG. 6): the probe is detached from the base and inserted into the food, causing the external charging voltage to disappear. The switching circuit is reset and connected to the interface multiplexing circuit at the back end. At this point, the electrode plates in the first half of the probe measure water and internal temperature, and the RF communication and environmental temperature measurement in the second half of the probe are fully operational under the scheduling of the time-sharing circuit.

In summary, the present disclosure provides a wireless food temperature probe with moisture detection function.

Firstly, this temperature probe is physically divided into two sections by insulating components, endowing the detection capacitor plates with electrical properties. Without adding any physical moisture sensors or internal wiring, accurate analysis of the moisture content and salinity inside food was achieved by reading the equivalent capacitance value of the dielectric between the electrode plates.

Secondly, the metal components at the tail of the probe are reused as wireless RF antennas, thermocouple temperature measurement circuit carriers, and external charging poles. In response to the problem of easy interference between high-frequency RF energy and low-frequency weak DC temperature difference signals, the present disclosure introduces an interface multiplexing circuit on the underlying hardware to achieve a physical isolation wall in the frequency band, and introduces a time-sharing circuit on the upper logic for millisecond level peak shifting scheduling. This dual insurance mechanism of resistance capacitance shunt and time-division interface multiplexing circuit effectively avoids electromagnetic crosstalk and achieves efficient, pure parallel acquisition and transmission of multi-source signals.

Thirdly, in response to the high voltage breakdown risk caused by the reuse of the tail metal component as charging poles, this embodiment has provided with a switching circuit at the entrance of the circuit board component. When the probe is connected to the external charging dock, the switching circuit instantly starts and forcibly disconnects all internal precision temperature measurement and communication links, greatly extending the service life and reliability of the probe.

The present disclosure provides a highly integrated sensing device through the combination of the above structure and circuit. It adopts the simplest five segment external physical topology and carries the most complex internal a plurality of signal branching and multiplexing logic, providing a new generation of preferred solutions for the field of intelligent kitchenware that combines compactness, safety, and multi-dimensional parameter detection capabilities.

It should be noted that when a component is referred to as “fixed” or “provided” on another component, it can be directly or indirectly provided on another component. When a component is referred to as “connected” to another component, it can be directly or indirectly connected to another component.

Besides that, terms “first” and “second” are only used for descriptive purposes and cannot be understood as indicating or implying relative importance or implying the number of technical features indicated. Thus, the features limited to “first” and “second” may explicitly or implicitly include one or more of these features. In the description of this application, the meanings of “a plurality of” and “several” refer to two or more, unless otherwise specified.

Note that the structure, proportion, size, etc. shown in the accompanying drawings of this specification are only for the purpose of cooperating with the content disclosed in the specification, for the understanding and reading of those familiar with this technology, and are not intended to limit the conditions that can be implemented in this application. Therefore, they do not have substantive technical significance. Any modification of the structure, change in proportion relationship, or adjustment of size should still fall within the scope of the technical content disclosed in this application without affecting the efficacy and purpose that can be achieved.

The above description of the disclosed embodiments enables those skilled in the art to implement or use the present disclosure. Various modifications to these embodiments will be apparent to those skilled in the art, and the general principles defined in this specification can be implemented in other embodiments without departing from the spirit or scope of the present disclosure. Therefore, the present disclosure will not be limited to the embodiments shown in this specification, but will conform to a widest scope consistent with the principles and novel features disclosed in this specification.

What is claimed is:

1. A wireless food temperature probe with moisture detection function, comprising: a probe body and a circuit board component that is provided in the probe body;
the probe body comprises a metal tip component, a metal tube component, and a first insulation connector; the metal tip component and the metal tube component are connected and electrically isolated through the first insulation connector;
the metal tip component is provided with a first temperature sensor configured to detect an internal temperature of food, and the first temperature sensor is electrically connected to the circuit board component;
the metal tip component and the metal tube component serve as two electrode plates for detecting capacitors, respectively;
the circuit board component is electrically connected to the metal tip component and the metal tube component, respectively; the circuit board component obtains moisture content of food by detecting a capacitance value between the metal tip component and the metal tube component.

2. The wireless food temperature probe with moisture detection function according to claim 1, wherein the circuit board component comprises a first temperature measurement module and a moisture detection module,
the first temperature sensor is electrically connected to the first temperature measurement module, and the moisture detection module is electrically connected to the metal tip component and the metal tube component respectively to obtain the moisture content of the food.

3. The wireless food temperature probe with moisture detection function according to claim 2, wherein the moisture detection module is further configured to obtain a salinity of the food based on the capacitance value.

4. The wireless food temperature probe with moisture detection function according to claim 1, wherein the probe body further comprises a second insulation connector and a metal tail component;
the metal tube component and the metal tail component are connected and electrically isolated through the second insulation connector.

5. The wireless food temperature probe with moisture detection function according to claim 4, wherein the metal tip component, the first insulation connector, the metal tube component, the second insulation connector, and the metal tail component are sequentially connected from front to back to form the probe body.

6. The wireless food temperature probe with moisture detection function according to claim 4, wherein the metal tail component is provided with a second temperature sensor configured to detect an ambient temperature, and the circuit board component further comprises a second temperature measurement module,
wherein the second temperature sensor is electrically connected to the second temperature measurement module.

7. The wireless food temperature probe with moisture detection function according to claim 6, wherein the metal tail component is configured as a second temperature sensor carrier, the second temperature sensor is a thermocouple,
a hot end of the thermocouple is connected to the metal tail component to render the metal tail component as a part of a thermocouple circuit, and a cold end of the thermocouple is connected to the second temperature measurement module of the circuit board component through a first wire and a second wire.

8. The wireless food temperature probe with moisture detection function according to claim 6, wherein the metal tail component is configured as an antenna for wireless signal transmission, and the circuit board component further comprises an Radio Frequency, (RF) main control chip and a time-sharing circuit, the time-sharing circuit is respectively electrically connected to the RF main control chip and the second temperature measurement module to achieve time-sharing processing of wireless signal transmission and environmental temperature acquisition.

9. The wireless food temperature probe with moisture detection function according to claim 6, wherein the metal tail component is configured as a part of an external charging circuit, and when charging externally, the metal tip component or the metal tube component serves as a first charging electrode of the external charging circuit, and the metal tail component serves as a second charging electrode of the external charging circuit, to charge a battery built into the probe body through the circuit board component.

10. The wireless food temperature probe with moisture detection function according to claim 8, wherein the circuit board component further comprises an interface multiplexing circuit;
the interface multiplexing circuit at least comprises an isolation capacitor and a RF choke inductor;
the RF main control chip is electrically connected to the metal tail component through the isolation capacitor, and the second temperature measurement module is electrically connected to the metal tail component through the RF choke inductor to achieve physical isolation between a RF signal and an environmental temperature acquisition signal.

11. The wireless food temperature probe with moisture detection function according to claim 9, wherein the circuit board component further comprises a charging module and a switching circuit,
the charging module is electrically connected to the battery;
the metal tail component is connected to the charging module through the switching circuit, and the switching circuit is configured to disconnect an electrical connection between the metal tail component with an internal temperature measurement path and a signal transmission path when the metal tail component is connected to an external power source.
